SEQUENCE LISTING (4 sequences)

SEQ ID NO: 1 - DNA, 50 bp, Lambda Bacteriophage
cccccctcactagtcatcaacccgtgagcttggacagccaatggttatac

SEQ ID NO: 2 - DNA, 50 bp, Lambda Bacteriophage
cccccctcactagtcatctgattccaggctttggctttagccgcttcggt

SEQ ID NO: 3 - DNA, 50 bp, Lambda Bacteriophage
cccccctcactagtcatctgattccaggctttggctttagccgcttcggg

SEQ ID NO: 4 - DNA, 50 bp, Lambda Bacteriophage
cccccctcactagtcatctgattccaggctttggctttagccgcttcgcg

(19) United States
(12) Patent Application Publication
(10) Pub. No.: US 2008/0118917 A1
(43) Pub. Date: May 22, 2008

(54) Isothermal SNP Detection Method

(75) Inventors: Christine D. Hardy, Hayward, CA (US); Patrick K. Martin, Redwood City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/562,345

(22) Filed: Nov. 21, 2006

(51) Int. Cl. C12Q 1/68; C07H 21/04

(52) U.S. Cl. 435/6; 536/23.1

(57) ABSTRACT

In some embodiments, the present teachings provide a method for detecting a nucleotide of interest comprising; forming an amplification reaction mixture comprising a mismatched primer, a target polynucleotide, a strand-displacing polymerase lacking 3′ to 5′ exonuclease activity, a recombinase, and a single-stranded DNA binding protein; hybridizing a mismatched primer to the target polynucleotide to form a primer-target complex; and, detecting the nucleotide of interest by the absence of a primer extension product. In some embodiments, control reactions are performed in which a control polynucleotide is exponentially amplified. Additional methods, as well as reaction mixtures and kits, are also provided.

### FIELD

The present teachings relate generally to molecular biology, and in particular to methods, compositions, and kits for detecting single nucleotide polymorphisms using isothermal amplification.

### INTRODUCTION

Amplification of nucleic acids is an important tool in molecular biology, medical diagnostics, and forensics. The most common method of nucleic acid amplification is the polymerase chain reaction (PCR), see for example U.S. Pat. No. 4,965,188, U.S. Pat. No. 4,683,202, and U.S. Pat. No. 4,683,195. Currently employed, PCR uses thermal cycling in combination with thermophilic polymerases to amplify nucleic acids. PCR thus employs a machine (a thermal cycler) that carefully modulates the temperature of the sample and a power source to run this machine.

Isothermal amplification describes a process that allows nucleic acids to be copied repeatedly without the need for temperature variation of the sample, thus obviating the need for a thermal cycler. One isothermal amplification procedure is recombinase polymerase amplification (RPA) (Piepenburg et al., PLOS Biology, July 2006, volume 4, issue 7, 1115-1121, and U.S. Patent Application US2005/0112631). RPA employs a recombinase, such as RecA protein from E. coli or the UvsX protein from phage T4, to mediate primer hybridization in the absence of heat denaturation of the template. The method also employs a DNA single strand binding protein, a mesophilic (or thermophilic) polymerase, a creatine kinase (for ATP regeneration) and factors believed to promote recombinase loading (crowding agents such as PEG and/or recombinase loader proteins.

Single nucleotide polymorphisms (SNPs) are single nucleotide differences between related genomes. SNPs can be used to differentiate individuals, to establish genetic linkage patterns, to test for cancer or other disease predispositions, and potentially, to predict drug sensitivities in patients. SNP detection can be carried out by a variety of methods, including Sanger-type DNA sequencing, the ligase detection reaction (LDR), the ligase chain reaction (LCR), flap-LCR (see U.S. Pat. No. 6,511,810, and 5′ nuclease PCR (TaqMan). However, these methods involve thermal cycling, and its attendant costs and equipment.

### SUMMARY

Additional methods, as well as reaction mixtures and kits, are also provided.

### DRAWINGS

FIG. 1 depicts an illustrative schematic according to some embodiments of the present teachings

FIG. 2 depicts some illustrative data according to some embodiments of the present teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### SOME DEFINITIONS

As used herein, the term “matched primer” refers to a primer that is complementary to a corresponding target polynucleotide, and does not contain a mismatch at its 3′ end. Typically, the matched primer will be completely complementary to the target polynucleotide, though slight mismatches away from the final nucleotide of the 3′ end that do not significantly interfere with hybridization may be tolerated, and are within the scope of the present teachings.

As used herein, the term “mismatched primer” refers to a primer that hybridizes partially to its corresponding target polynucleotide. A mismatched primer contains a complementary portion, and a non-complementary portion that is not complementary to the target polynucleotide. The non-complementary portion of the mismatched primer is located at its 3′ end, fails to hybridize with a nucleotide of interest present in a target polynucleotide, and is typically one nucleotide in length. In some embodiments, there can also be other destabilizing mismatches near the 3′ end to increase specificity of the primer hybridization reaction. Typically, the complementary portion of the mismatched primer will be completely complementary to the target polynucleotide, though slight mismatches that do not significantly interfere with hybridization may be tolerated, and are within the scope of the present teachings. The complementary portion can be any appropriate length. In some embodiments, the complementary portion is at least 6, at least 7, at least 8, at least 9, at least 10, at least 11, at least 12, at least 13, at least 14, at least 15, at least 15, at least 16, at least 17, at least 18, at least 19, or greater than 20 nucleotides in length.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include oligonucleotide synthesis, and hybridization (including recombinase-mediated hybridization), extension reactions, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The primers of the present teachings can employ nucleotides as well as nucleotide analogs, including synthetic analogs having modified nucleoside base moieties, modified sugar moieties, and/or modified phosphate groups and phosphate ester moieties. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures:

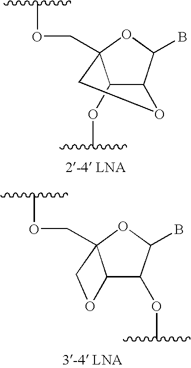

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

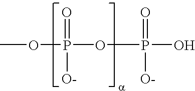

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The present teachings provide a novel isothermal amplification process for detecting SNPs. In the process of performing primer specificity studies, the inventors made two observations. First, as expected, Taq Polymerase, which as no 3′-5′ exonuclease activity, produced relatively little PCR product using a mismatched primer that contained a single nucleotide mismatch at its 3′ end, and produced no product when using a primer with two nucleotide mismatches at its 3′ end. Second, comparing these PCR results to isothermal amplification results showed that isothermal amplification does not exhibit a higher tolerance to internal and 3′ mismatches than PCR. These two observations led to the hypothesis that an isothermal amplification procedure could be employed to detect SNPs. Such an approach is based on the inability of polymerases lacking a 3′ to 5′ exonuclease to excise a mismatched base located at the 3′ end of a primer, thus preventing primer extension.

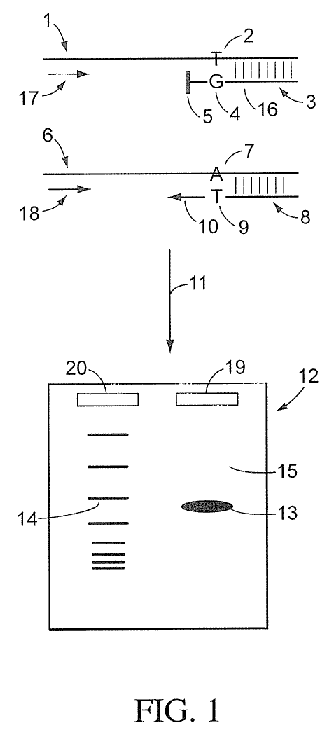

An illustrative schematic of an isothermal amplification reaction employed to detect a SNP is depicted in FIG. 1. Here, a first target polynucleotide (1) containing nucleotide of interest (T, 2) is queried with a reverse mismatch primer (3) and a forward matched primer (17). The reverse mismatch primer contains a complementary portion (16) and a non-complementary portion (4, here a G). As a result of the absence of 3′-5′ exonuclease activity of the polymerase, extension of the mismatched primer is inhibited (5). A 37 C incubation for 2 hours along with a recombinase, a DNA single-stranded binding protein, and a polymerase lacking 3′-5′ exonuclease activity, can then be performed. Thereafter, running an aliquot of the resulting reaction products (11) on a mobility dependant analysis technique such as an electrophorietic gel (12), results in the absence of a corresponding band (15) of the appropriate size by reference to a size ladder (14). To verify that the absence of a band reflects the presence of the T (2) nucleotide of interest in the target polynucleotide, rather than failure of the reaction for other reasons, a parallel control reaction can be performed. Here for example, a control polynucleotide (6) is queried with a reverse matched primer (8) and a forward matched primer (18). The 3′ end of the reverse matched primer contains a T (9), and is complementary to the nucleotide of interest (A, 7) in the control polynucleotide. As a result of the presence of complementarity at the 3′ end of the reverse matched primer with the control target polynucleotide, no excision of a mismatched base is needed. Correspondingly, the absence of 3′-5′ exonuclease activity of the polymerase does not prevent extension of the matched primer. Thus, extension occurs, resulting in an amplification product. Running an aliquot of the resulting reaction products on an agarose gel (12) results in the presence of a corresponding band (13) of the appropriate size by reference to a size ladder (14). Such a control reaction thus verifies that the absence of product (15) results from the presence of a T nucleotide of interest in the first target polynucleotide. In some embodiments, (1) and (6) can be the same target region, if for example one used different reverse primers in each reaction, each representing one allele of the same SNP. Discrimination using a mobility dependant analysis technique such as capillary electrophoresis can be achieved by differentially labeling of the reverse primers, for example with different flourophores, and/or with reverse primers bearing different lengths.

An example to demonstrate this approach was performed. Paired isothermal amplification reactions were performed containing a total reaction volume of 30 ul, with 6 ug RecA protein (USB), 25 U polymerase (Klenow, Klenow exo⁻ polymerase, or E. coli Pol I, as indicated), 250 nM each primer, 5% PEG compound (Sigma P2263), ˜15 ng restriction digested Lambda phage DNA, 3 mM ATP, 200 uM dNTPs, 1 U creatine kinase (2.7 ug), 30 mM creatine phosphate, 50 mM Tris (pH 7.4), 50 mM K(OAc), 10 mM Mg (OAc)₂, and 5 mM DTT. The reactions were incubated for 2 hours at 37 C after which they underwent phenol:chloroform extraction followed by ethanol precipitation of the aqueous layer. One half of the recovered material was loaded on the gels.

Sequences of the primers employed, with mismatches shown in bold, were:

SEQ ID NO: 1

EcoRV fwd
CCCCCCTCACTAGTCATCAACCCGTGAGCTTGGACAGCCAATGGTTATAC

SEQ ID NO: 2

EcoRV rev
CCCCCCTCACTAGTCATCTGATTCCAGGCTTTGGCTTTAGCCGCTTCGGT

SEQ ID NO: 3

EcoRV rev 1MM
CCCCCCTCACTAGTCATCTGATTCCAGGCTTTGGCTTTAGCCGCTTGGGG

SEQ ID NO: 4:

EcoRV rev 2MM
CCCCCCTCACTAGTCATCTGATTCCAGGCTTTGGCTTTAGCCGCTTCGCG

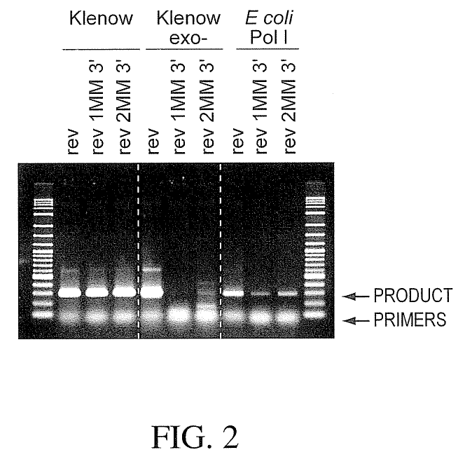

The results of this experiment are shown in FIG. 2. These data indicate that both Klenow and E. coli Pol I (which both have 3′-5′ exo activities) produced amplification product when 3′ mismatched primers were used, whereas Klenow exo⁻ polymerase did not produce significant amounts of product when one or two mismatches were placed at the 3′ end of one primer (FIG. 2, where “1 MM” indicates one mismatched nucleotide and “2 MM” indicates two mismatched nucleotides).

Thus, in some embodiments the present teachings provide a method for detecting a nucleotide of interest comprising; providing a mismatched primer comprising a complementary portion and a non-complementary portion; hybridizing the complementary portion of the mismatched primer to a target polynucleotide to form a primer-target complex in an amplification reaction mixture; and, detecting the nucleotide of interest by the absence of a primer extension product, with the proviso that temperature cycling is not performed.

In some embodiments, the present teachings provide a method for detecting a nucleotide of interest by the absence of a primer extension product comprising; forming an amplification reaction mixture comprising a mismatched primer, a target polynucleotide, a strand-displacing polymerase lacking 3′ to 5′ exonuclease activity, a recombinase, and a single-stranded DNA binding protein; hybridizing a mismatched primer to the target polynucleotide to form a primer-target complex; and, detecting the nucleotide of interest by the absence of a primer extension product.

In some embodiments, the present teachings provide a method for detecting a nucleotide of interest in a first target polynucleotide comprising; forming an amplification reaction mixture comprising a mismatched primer, a matched primer, a first target polynucleotide, a second target polynucleotide, a strand-displacing polymerase lacking 3′ to 5′ exonuclease activity, a recombinase, and a single-stranded DNA binding protein; hybridizing the mismatched primer to the first target polynucleotide to form

a first primer-target complex and hybridizing the matched primer to the second target polynucleotide to form a second primer-target complex; and, measuring the absence of a first amplification product from the first target polynucleotide and the presence of a second amplification product from the second target polynucleotide to detect the nucleotide of interest in the first target polynucleotide.

In some embodiments, the present teachings provide a method for detecting a nucleotide of interest in a first target polynucleotide comprising; forming a first amplification reaction mixture comprising a mismatched primer, a first target polynucleotide, a strand-displacing polymerase lacking 3′ to 5′ exonuclease activity, a recombinase, and a single-stranded DNA binding protein; forming a second amplification reaction mixture comprising a matched primer, a second target polynucleotide, a strand-displacing polymerase lacking 3′ to 5′ exonuclease activity, a recombinase, and a single-stranded DNA binding protein; hybridizing the mismatched primer to the first target polynucleotide to form a first primer-target complex and hybridizing the matched primer to the second target polynucleotide to form a second primer-target complex; and, measuring the absence of a first amplification product from the first target polynucleotide in the first amplification reaction mixture and the presence of a second amplification product from the second target polynucleotide in the second amplification reaction mixture to detect the nucleotide of interest in the first target polynucleotide.

In some embodiments, the present teachings provide a method of preventing amplification of a first target polynucleotide while allowing amplification of a second target polynucleotide comprising; forming an amplification reaction mixture comprising a mismatched primer, a matched primer, a first target polynucleotide, a second target polynucleotide, a strand-displacing polymerase lacking 3′ to 5′ exonuclease activity, a recombinase, and a single-stranded DNA binding protein; hybridizing the mismatched primer to the first target polynucleotide to form a first primer-target complex and hybridizing the matched primer to the second target polynucleotide to form a second primer-target complex; and, preventing amplification of the first target polynucleotide while allowing amplification of the second target polynucleotide, with the proviso that the amplification is isothermal.

Any of a variety of recombinases can be used in the context of the present teachings. For example, in some embodiments the recombinase is selected from the group consisting of RecA-type proteins from bacteriophage and Prokaryotes (e.g. UvsX from bacteriophage T4, recA from E. coli); orthologs from Archaebacteria (RadA/RadB type); and orthologs from Eukaryotes (Rad51, DMC1, and Rad55/57 type proteins).

Any of a variety of single-stranded DNA binding proteins can be used in the context of the present teachings. For example, in some embodiments the single-stranded DNA binding protein is selected from the group consisting of bacteriophage SSBs (e.g. T4 gp32, T7 gene 2.5 (and mutants thereof), and Phi 29 gene 5), bacterial SSBs (e.g. E. coli ssb), the replication protein A (RPA) orthologs from Eukaryotes, and similar Archaeal proteins (e.g. ssb from Aeropyrum pernix, and RPA from Pyrococcus furiosus).

Any of a variety of strand-displacing polymerase lacking 3′ to 5′ exonuclease activity can be used in the context of the present teachings. For example, in some embodiments the strand-displacing polymerase lacking 3′ to 5′ exonuclease activity is selected from the group consisting of A-family polymerases also lacking intrinsic 5′ to 3′ exonuclease activity (e.g. appropriately modified versions of E. coli Pol I (Klenow exo⁻), and similarly modified polymerases from organisms such as B. stearothermophilus (B. ste exo-), Thermus aquaticus (Thermosequenase, AmpliTaq CS, AmpliTaq FS) and Aquifex aeolicus, bacteriophage T5, and bacteriophage T7 (Sequenase), and B-family polymerases (Phi29 exo-, Vent pot exo-, Thermococcus spp. 9° Nₘ exo-).

In some embodiments, a crowding agent can be employed. Without intending to be limited to any particular theory, crowding agents are believed to promote recombinase loading, thus establishing favorable reaction conditions. Exemplary crowding agents include carbowax20M, other PEG compounds, as well as recombinase loading factors such as T4 UvsY, Rb 69 UvsY, E. coli Rec OR.

While the present teachings are well-suited to the detection of naturally occurring SNPs, other applications are also envisioned. For example, the epigenomic phenomenon of cytosine methylation can be explored by application of the present teachings. Here, un-methylated cytosines can be converted to uracil through the action of bisulfite. Such conversion results in a nucleotide change that can be queried with the isothermal amplification procedures of the present teachings by design of a mismatched primer with a 3′ mismatch nucleotide corresponding to the site of a cytosine. Illustrative bisulfite treatment methods and additional information on the study of methylation generally can be found in Boyd et al., Anal Biochem. 2004 Mar. 15; 326(2):278-80; Boyd et al., Anal Biochem. 2006 Jul. 15; 354(2):266-73 Epub 2006 May 6, as well as U.S. patent application Ser. No. 11/424,224, U.S. patent application Ser. No. 11/352,143, and U. S. patent application Ser. No. 10/926,531.

In some embodiments, differences in splice variants between mRNAs can be queried according to the present teachings. For example, following conversion of mRNA to cDNA by reverse transcription, primers can designed to query sequence differences corresponding to splice variants.

In some embodiments, the present teachings provide a reaction mixture comprising; a first composition comprising a first target polynucleotide hybridized to a complementary portion of a mismatch primer, wherein the mismatch primer further comprises a one nucleotide non-complementary portion at its 3′ end; a second composition comprising a second target polynucleotide hybridized to a matched primer; and, a strand-displacing polymerase lacking 3′ to 5′ exonuclease activity, a recombinase, and a single-stranded DNA binding protein.

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

Thus, in some embodiments the present teachings provide a kit comprising; a matched primer and a mismatched primer, wherein the matched primer and the mismatched primer differ only in the nucleotide at their 3′ ends; and, a strand-displacing polymerase lacking 3′ to 5′ exonuclease activity, a recombinase, and a single-stranded DNA binding protein.

Of course, in some embodiments the reaction mixture, and/or kits, can further comprise any of the species of recombinases, strand-displacing polymerases lacking 3′ to 5′ exonuclease activity, and/or single-stranded DNA binding proteins described supra. The reaction mixture and/or kits can also comprise at least one matched primer, at least one mismatched primer, nucleotides, labels (such as SYBR Green for example), buffers, size standards, or any combination thereof.

Detection of the amplification products can employ any of a variety of detection systems. For example, as depicted in FIGS. 1 and 2, conventional agarose gel electrophoresis can be employed using an inter-chelating dye such as ethidium bromide. In other embodiments, detector probes such as double-stranded DNA specific probes such as SYBR Green can be employed, and detection achieved by fluorescent measurements. In general, the detection of the resulting isothermal amplification products can be achieved by any tool readily available to the contemporary molecular biologist, and are not a limitation of the present teachings. Such tools include mobility dependant analysis techniques such as capillary electrophoresis and mass spec, electrochemical detection, and the use of Taq-Man like probes.

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method for detecting a nucleotide of interest by the absence of a primer extension product comprising;
forming an amplification reaction mixture comprising a mismatched primer, a target polynucleotide, a strand-displacing polymerase lacking 3′ to 5′ exonuclease activity, a recombinase, and a single-stranded DNA binding protein;
hybridizing a mismatched primer to the target polynucleotide to form a primer-target complex; and,
detecting the nucleotide of interest by the absence of a primer extension product.

2. A method for detecting a nucleotide of interest in a first target polynucleotide comprising;
forming an amplification reaction mixture comprising a mismatched primer, a matched primer, a first target polynucleotide, a second target polynucleotide, a strand-displacing polymerase lacking 3′ to 5′ exonuclease activity, a recombinase, and a single-stranded DNA binding protein;
hybridizing the mismatched primer to the first target polynucleotide to form a first primer-target complex and hybridizing the matched primer to the second target polynucleotide to form a second primer-target complex; and,
measuring the absence of a first amplification product from the first target polynucleotide and the presence of a second amplification product from the second target polynucleotide to detect the nucleotide of interest in the first target polynucleotide.

3. A method for detecting a nucleotide of interest in a first target polynucleotide comprising;
forming a first amplification reaction mixture comprising a mismatched primer, a first target polynucleotide, a strand-displacing polymerase lacking 3′ to 5′ exonuclease activity, a recombinase, and a single-stranded DNA binding protein;
forming a second amplification reaction mixture comprising a matched primer, a second target polynucleotide, a strand-displacing polymerase lacking 3′ to 5′ exonuclease activity, a recombinase, and a single-stranded DNA binding protein;
hybridizing the mismatched primer to the first target polynucleotide to form a first primer-target complex and hybridizing the matched primer to the second target polynucleotide to form a second primer-target complex; and,
measuring the absence of a first amplification product from the first target polynucleotide in the first amplification reaction mixture and the presence of a second amplification product from the second target polynucleotide in the second amplification reaction mixture to detect the nucleotide of interest in the first target polynucleotide.

4. A method of preventing amplification of a first target polynucleotide while allowing amplification of a second target polynucleotide comprising;
forming an amplification reaction mixture comprising a mismatched primer, a matched primer, a first target polynucleotide, a second target polynucleotide, a strand-displacing polymerase lacking 3′ to 5′ exonuclease activity, a recombinase, and a single-stranded DNA binding protein;
hybridizing the mismatched primer to the first target polynucleotide to form a first primer-target complex and hybridizing the matched primer to the second target polynucleotide to form a second primer-target complex; and,
preventing amplification of the first target polynucleotide while allowing amplification of the second target polynucleotide, with the proviso that the amplifying is isothermal.

5. The method of any of claims 1-4 wherein the recombinase is selected from the group consisting of bacteriophage T4 UvsX, E. coli recA, Archaebacteria RadA, Archaebacteria RadB, eurkaryotic Rad51, eukaryotic DMC1, and eukaryotic Rad55/57.

6. The method of any of claims 1-4 wherein the single-stranded DNA binding protein is selected from the group consisting of bacteriophage T4 gp32, bacteriophage T7 gene 2.5, Phi 29 gene 5, E. coli ssb, eukaryotic RPA, Aeropyrum pernix ssb, and Pyrococcus furiosus RPA.

7. The method of any of claims 1-4 wherein the strand-displacing polymerase lacking 3′ to 5′ exonuclease activity is selected from the group consisting of Klenow exo⁻, B. stearothermophilus exo-, Thermus aquaticus polymerase, Aquifex aeolicus polymerase, bacteriophage T5 polymerase, bacteriophage T7 polymerase, Phi29 exo-, Vent pol exo-, and Thermococcus spp. 9° Nₘ exo-.

8. The method of any of claims 1-4 further comprising a crowding agent.

9. The method of claim 8 wherein the crowding agent is selected from the group consisting of carbowax20M, a PEG compound, T4 UvsY, Rb 69 UvsY, and E. coli Rec OR.

10. A reaction mixture comprising;
a first composition comprising a first target polynucleotide hybridized to a complementary portion of a mismatch primer, wherein the mismatch primer further comprises a one nucleotide non-complementary portion at its 3′ end;
a second composition comprising a second target polynucleotide hybridized to a matched primer; and,
a strand-displacing polymerase lacking 3′ to 5′ exonuclease activity, a recombinase, and a single-stranded DNA binding protein.

11. The reaction composition according to claim 10 wherein the recombinase is selected from the group consisting of bacteriophage T4 UvsX, E. coli recA, Archaebacteria RadA, Archaebacteria RadB, eurkaryotic Rad51, eukaryotic DMC1, and eukaryotic Rad55/57.

12. The reaction composition according to claim 10 wherein the single-stranded DNA binding protein is selected from the group consisting of bacteriophage T4 gp32, bacteriophage T7 gene 2.5, Phi 29 gene 5, E. coli ssb, eukaryotic RPA, Aeropyrum pernix ssb, and Pyrococcus furiosus RPA.

13. The reaction composition of claim 10 wherein the strand-displacing polymerase lacking 3′ to 5′ exonuclease activity is selected from the group consisting of Klenow exo-, B. stearothermophilus exo-, Thermus aquaticus polymerase, Aquifex aeolicus polymerase, bacteriophage T5 polymerase, bacteriophage T7 polymerase, Phi29 exo-, Vent pol exo-, and Thermococcus spp. 9° Nₘ exo-.

14. The reaction composition according to claim 10 further comprising a crowding agent.

15. The reaction composition according to claim 14 wherein the crowding agent is selected from the group consisting of carbowax20M, a PEG compound, T4 UvsY, Rb 69 UvsY, and E. coli Rec OR.

16. A kit comprising;
a matched primer and a mismatched primer, wherein the matched primer and the mismatched primer differ only in the nucleotide at their 3′ ends; and,
a strand-displacing polymerase lacking 3′ to 5′ exonuclease activity, a recombinase, and a single-stranded DNA binding protein.

17. The kit according to claim 16 wherein the recombinase is selected from the group consisting of bacteriophage T4 UvsX, E. coli recA, Archaebacteria RadA, Archaebacteria RadB, eurkaryotic Rad51, eukaryotic DMC1, and eukaryotic Rad55/57.

18. The kit according to claim 16 wherein the single-stranded DNA binding protein is selected from the group consisting of bacteriophage T4 gp32, bacteriophage T7 gene 2.5, Phi 29 gene 5, E. coli ssb, eukaryotic RPA, Aeropyrum pernix ssb, and Pyrococcus furiosus RPA.

19. The kit according to claim 16 wherein the strand-displacing polymerase lacking 3′ to 5′ exonuclease activity is selected from the group consisting of Klenow exo-, B. stearothermophilus exo-, Thermus aquaticus polymerase, Aquifex aeolicus polymerase, bacteriophage T5 polymerase, bacteriophage T7 polymerase, Phi29 exo-, Vent pol exo-, and Thermococcus spp. 9° Nₘ exo-.

20. The kit according to claim 16 further comprising a crowding agent.

21. The reaction composition according to claim 20 wherein the crowding agent is selected from the group consisting of carbowax20M, a PEG compound, T4 UvsY, Rb 69 UvsY, and E. coli Rec OR.
